(19) United States
(12) Patent Application Publication
(10) Pub. No.: US 2019/0351169 A1
(43) Pub. Date: Nov. 21, 2019

(54) RESPIRATORY AIRWAY ASSISTIVE DEVICE

(71) Applicant: Jennie Soi Phui, Newport Beach, CA (US)

(72) Inventor: Jennie Soi Phui, Newport Beach, CA (US)

(21) Appl. No.: 15/981,730

(22) Filed: May 16, 2018

(51) Int. Cl. A61M 16/06; A61M 16/00

(52) U.S. Cl. CPC A61M 16/0605; A61M 16/0003; A61M 2205/581; A61M 2210/0625; A61M 2230/20; A61M 2230/50

(57) ABSTRACT

Using various embodiments, methods and systems for a respiratory airway assistive device are described. In one embodiment, the device includes an airway maintenance body comprising an dorsal surface and an ventral surface, a first end, a first aperture having a first opening positioned at or near the first terminus, a second end, a second aperture positioned at or near the second terminus and that is in fluidic communication with the first aperture, wherein the first end is configured to be accommodated inside the mouth of a human subject, and wherein the second end is configured to be outside the mouth, and wherein the first end includes a curvature, the curvature at the ventral surface accommodating a cavity. The device further includes a vestibular guard coupled to the airway maintenance body forming an occlusal rest portion on the dorsal surface or the airway maintenance body.

### CROSS-REFERENCE TO RELATED APPLICATIONS

The present application is related to U.S. Design patent application Ser. No. ______, concurrently filed herewith, and titled “Respiratory Airway Assistive Device.” The contents of the above stated matter are incorporated herein by reference for all purposes to the extent that such subject matter is not inconsistent herewith or limiting hereof.

### FIELD OF THE INVENTION

Embodiments of the present invention relates generally to the field of prevention of respiratory airway obstruction. More particularly, embodiments of the invention relate to a device to assist in respiration by preventing the collapse of the upper airway.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Current devices on the market are inadequate in providing respiratory assistance effectively for sleep related disorders. As a result, there is a need for a device that effectively provides respiratory airway assistance.

### SUMMARY OF THE DESCRIPTION

Embodiments of the present invention are best understood by reference to the detailed figures and description set forth herein. Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive. Compounds, materials, manufacturing techniques, uses, and applications, described herein, as these may vary.

It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. It is to be further understood that the present invention is not limited to the particular methodology in the art. Similarly, for another example, a reference to “a step” or “a means” is a reference to one or more steps or means and may include sub steps and subservient means.

All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

One general aspect includes a respiratory airway assistive device, including: an airway maintenance body including an dorsal surface and an ventral surface, a first end, a first aperture having a first opening positioned at or near the first end, a second end, a second aperture positioned at or near the second end and that can be in communication with the first aperture via a fluidic communication channel, where the first end can be configured to be accommodated inside the oral cavity of a human subject, and where the second end can be configured to be outside the oral cavity, and where the first end includes a curvature, the curvature at the ventral surface accommodating a cavity; and a vestibular guard coupled to the airway maintenance body forming an occlusal rest portion on the dorsal surface or the airway maintenance body. The respiratory airway assistive device can be constructed with a plaint material.

Implementations may include one or more of the following features: The respiratory airway maintenance device can have a dorsal surface that comes in direct contact with a palate of the oral cavity of the human subject at a superior end. The airway maintenance device can also come in direct contact with a floor of the oral cavity at an inferior end. The respiratory airway maintenance device can be configured to have an intraoral cavity that can be segmented into an upper intraoral space and a lower intraoral space. The upper intraoral space can accommodate the first aperture and provide an open airway to assist in an obstruction free fluidic communication with the second aperture, and the lower intraoral space can be configured as a tongue stabilizer to accommodate the tongue of the human subject.

The respiratory airway maintenance device can further include a temperature sensor. The temperature sensor can be a sublingual thermometer. The respiratory airway maintenance device can further include a vitals monitoring sensor to monitor the vitals of the human subject. The vitals monitoring sensor can be configured to be located on a surface of the vestibular guard or any other surface, as known to a person of ordinary skill in the art, from where vitals can be measured.

The respiratory airway maintenance device can further include a lip guard coupled to the airway maintenance body forming a lip rest portion on the dorsal surface of the airway maintenance body. The dorsal surface of the lip guard can be configured to include at least one of a vitals display, temperature display, and a timer. The respiratory airway assistive device can be configured to accommodate a retaining strap affixed to the lip guard.

In one or more embodiments, the fluidic communication channel can include a sound magnifier. The sound magnifier can be configured to be positioned near or at the second aperture within the fluidic communication channel. The respiratory airway assistive device can also include a pulse oximeter. The respiratory airway assistive device can also include a coupling that can be in fluidic communication with the second aperture. The coupling can be configured to mate with a ventilator or any external source. The airway maintenance body can be dimensioned to fit within the oral cavity of a human subject, including but not limited to a child of nursing age, an older child, or an adult. The configuration of the respiratory airway assistive device can be changed according to the age and requirements of the human subject as described further herein. In one of more embodiments, the pliant material using which the respiratory airway assistive device is constructed can be rubber, latex, polyurethane, vinyl, silicone rubber, nitrile, or a combination thereof. The materials can be of medical grade.

In one or more embodiments, the fluidic communication channel can be configured to provide a clear airway from the second aperture to the first aperture. The airway maintenance body can also include a flavorant. The dorsal surface of the respiratory airway maintenance body can be configured so as to not contact teeth or a gingiva of the oral cavity when in use. Implementations of the described techniques may include hardware, a method or process, or computer software on a computer-accessible medium.

### BRIEF DESCRIPTION OF THE DRAWINGS

The present invention is illustrated by way of example and not limitation in the figures of the accompanying drawings in which like references indicate similar elements.

FIG. 1 is an isometric view of a respiratory airway assistive device of the present invention shown attached to a person, according to one embodiment of the present invention.

FIGS. 2A and 2B are front and back isometric views, respectively, of the respiratory airway assistive device, according to one embodiment of the present invention.

FIG. 3 is a front view of the respiratory airway assistive device, according to one embodiment of the present invention.

FIG. 4 is a back view of the respiratory airway assistive device, according to one embodiment of the present invention.

FIG. 5 is a top view of the respiratory airway assistive device, according to one embodiment of the present invention.

FIG. 6 is a side view of the respiratory airway assistive device, according to one embodiment of the present invention.

FIG. 7 is a side view of an external connector for the respiratory airway assistive device, according to one embodiment of the present invention.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” or “another embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention.

Structures described herein are to be understood also to refer to functional equivalents of such structures. The present invention will now be described in detail with reference to embodiments thereof as illustrated in the accompanying drawings. From reading the present disclosure, other variations and modifications will be apparent to persons skilled in the art. Such variations and modifications may involve equivalent and other features which are already known in the art, and which may be used instead of or in addition to features already described herein. Although Claims may have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub combination. References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention.

A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Those skilled in the art will readily recognize, in light of and in accordance with the teachings of the present invention, that any of the foregoing steps may be suitably replaced; reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

The present invention will now be described in detail with reference to embodiments thereof as illustrated in the accompanying drawings.

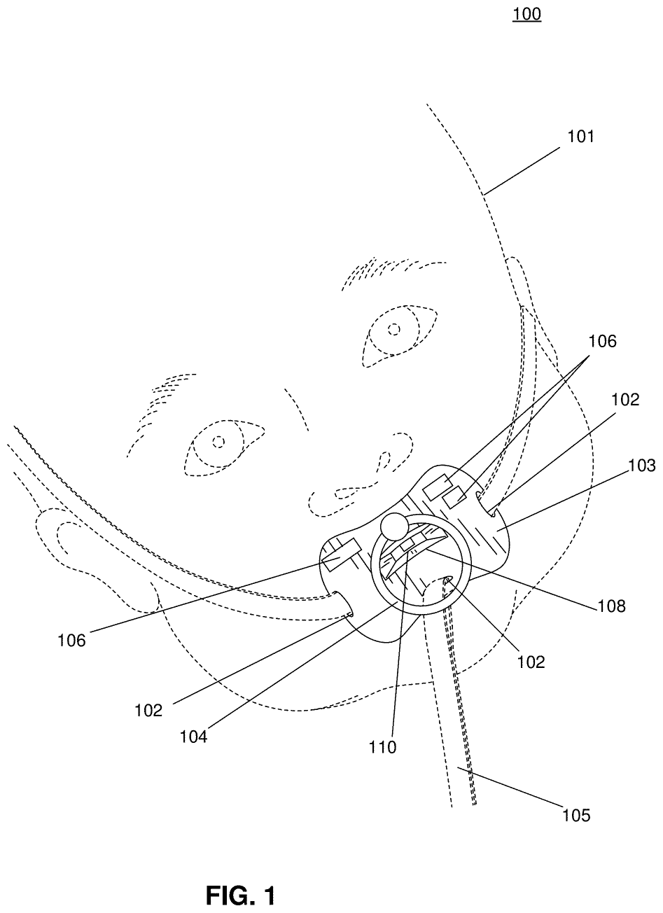

FIG. 1 shows respiratory airway assistive device 100 configured to assist user 101 in respiration (e.g., to prevent sleep apnea or other respiration related problems and issues). The dashed lines in this figure are used to illustrated unclaimed subject matter to illustrated the environment in which the invention may be used. Although user 101 is shown as a child, a person of ordinary skill in the art would appreciate that user 101 can be of any age and the techniques described herein are not limited to assist a user a particular age group and respiratory airway assistive device 100 can be configured accordingly. As illustrated, for a configuration involving a toddler, respiratory airway assistive device 100 can be configured to comprise an outer surface 103 having retaining perforation(s) 102 to retain/couple device 100 to user 101 via a strap. In one or more embodiments device 100 can also include safety ring 104 (coupled to outer surface 103) for easy removal of device 100 from user 101. In a configuration for adults or older children, in one embodiment, respiratory airway assistive device 100 can be configured without outer surface 103. Respiratory airway assistive device 100 can also include vitals monitor(s) display 106 that can display important vitals (e.g., temperature, heart rate, pulse, etc.) of user 101 when respiratory airway assistive device 100 is in use. respiratory airway assistive device 100 comprises at least one aperture 108 at a front terminus in communication via air (or fluidic) channel 110, to form a path of aeration, with an airway maintenance body (not shown) dimensioned to fit within an oral cavity of user 101 in need of airway assistance. Aperture 108 can be of any geometrical shape (e.g., circular, square, rectangular, triangular, rhomboid, or a combination thereof, etc.).

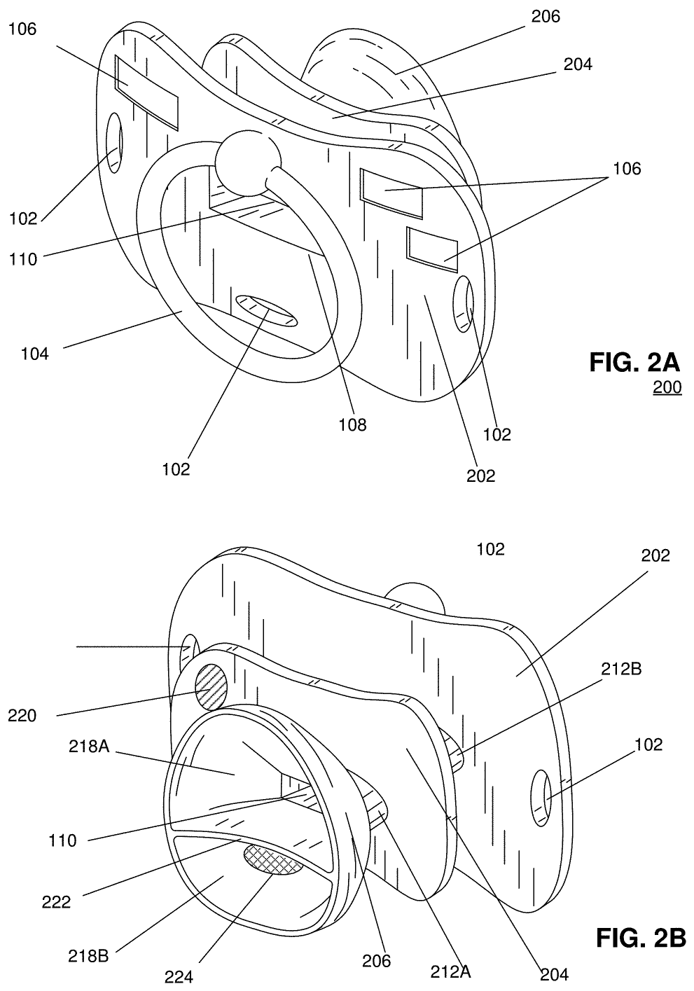

FIG. 2A illustrates a front isometric view of respiratory airway assistive device 100, according to one embodiment of the present invention. As illustrated, respiratory airway assistive device 100 can be configured such that outer surface 103 can be lip guard 202. In one or more embodiments, respiratory airway assistive device 100 can also be configured to comprise vestibular guard 204 and airway maintenance body 206. FIG. 2B illustrates a back isometric view of respiratory airway assistive device 100, according to one embodiment of the present invention. As illustrated, airway maintenance body 206 of respiratory airway assistive device 100 includes a back terminus 208, a back aperture 210 positioned at or near back terminus 208. Aperture 210 can be of any geometrical shape (e.g., circular, square, rectangular, triangular, rhomboid, or a combination thereof, etc.). Aperture 210 can be in fluidic communication with the front aperture 108 via fluidic communication channel 110.

In one embodiment, fluidic communication channel 110 forms a path of aeration. Airway maintenance body 206 includes outer (superior/dorsal) surface 214 and inner (inferior/ventral) surface 216. Outer surface 214 can be configured to have a first curvature dimensioned to contact the palate of the oral cavity at the top (superior end) and the floor (inferior end) of the oral cavity of user 101 when respiratory airway assistive device 100 is in use. In one embodiment, airway maintenance body 206 can be constructed of a pliant material. Outer surface 214, at the superior end of airway maintenance body 206, can be configured to provide palatal support to user 101 as illustrated.

In one embodiment, inner surface 214 of airway maintenance body 206 of respiratory airway assistive device 100 includes intraoral space that includes access to fluidic communication channel 110. In one embodiment, intraoral space can be configured to be divided into upper intraoral space 218A and lower intraoral space 218B via divider 222. In one embodiment, upper intra oral space 218A accommodates aperture 210 and fluidic communication channel 110.

In one embodiment, lower intra oral space 218B can be configured to accommodate a tongue stabilizer module to accommodate the tongue of user 101. Lower intraoral space 218B can include sublingual temperature sensor 224 (e.g., thermometer) to monitor the sublingual temperature of user 101. In yet another embodiment, aperture 210 is characterized in having an opening that is at least 50% or greater to the opening of aperture 108. In one embodiment, respiratory airway assistive device 100 can also include vitals sensor 220 (e.g., sensor to measure heart-rate, pulse oximeter, etc.). In yet another embodiment, vitals sensor 220 can also include a temperature sensor. Thus, in this embodiment, temperature sensor 224 may not be needed or can be replaced with another sensor, as appreciated by a person of ordinary skill in the art. A person or ordinary skill in the art would appreciate that the location of vitals sensor 220 and/or temperature sensor 224 can be adjusted as appropriate. In one embodiment, vitals display 106 can be coupled to vitals sensor 220 and/or temperature sensor 224 to display the vitals (e.g., heart-rate, pulse rate, temperature, etc.).

In another embodiment, vitals display 106 can also include a timer that can be configured to use as a dosage timer (e.g., for use as a timer to count the dosage of any prescription medication or for any other person, as determined to be appropriate by a person having ordinary skill in the art). respiratory airway assistive device 100 can also include a wireless transmitting module/device to transmit data generated by any sensor to an external computing device (e.g., computer, mobile device, etc.). Further, in embodiments for older children and adults (without retainer disc/lip guard 202), vitals display can be absent. In such an embodiment, any sensors on respiratory airway assistive device 100 can transmit reading directly to the external computing device.

As illustrated in FIG. 2A and FIG. 2B, respiratory airway maintenance device 100 includes a vestibular guard 204 coupled to airway maintenance body 206 via an outer surface of fluidic communication channel 110. In one embodiment an outer surface of the fluidic channel 110, between vestibular guard 204 and airway maintenance body 206, can be configured as an occlusal rest 212A. Lip guard 202 can be coupled to vestibular guard 204 via fluidic communication channel 110, as illustrated. A portion of fluidic communication channel between vestibular guard 204 and lip guard 202 can be configured as an lip rest section 212B on a dorsal surface. In one or more implementations lip guard 202 can also be configured as a retaining disc coupled at or near the front terminus of respiratory airway assistive device 100 comprising aperture 110. In this embodiment, retainer disc can have perforation(s) 102 to couple respiratory airway assistive device 100 with retainer straps 105 to user 101.

In one or more implementations, airway maintenance body 206 can also comprise fluidic communication channel 110 that is in fluid communication with the aperture 110 and aperture 210. In this embodiment, fluidic communication channel 110 is characterized by a first volume and the intraoral space (collectively represented by 218A and 218B) of airway maintenance body 206 is characterized by a second volume, the first volume being at least 50% of the second volume. In another embodiment, retaining disc/lip guard 202 is configured to be larger than intraoral cavity to prevent lip guard from being accommodated within the oral cavity of user 101.

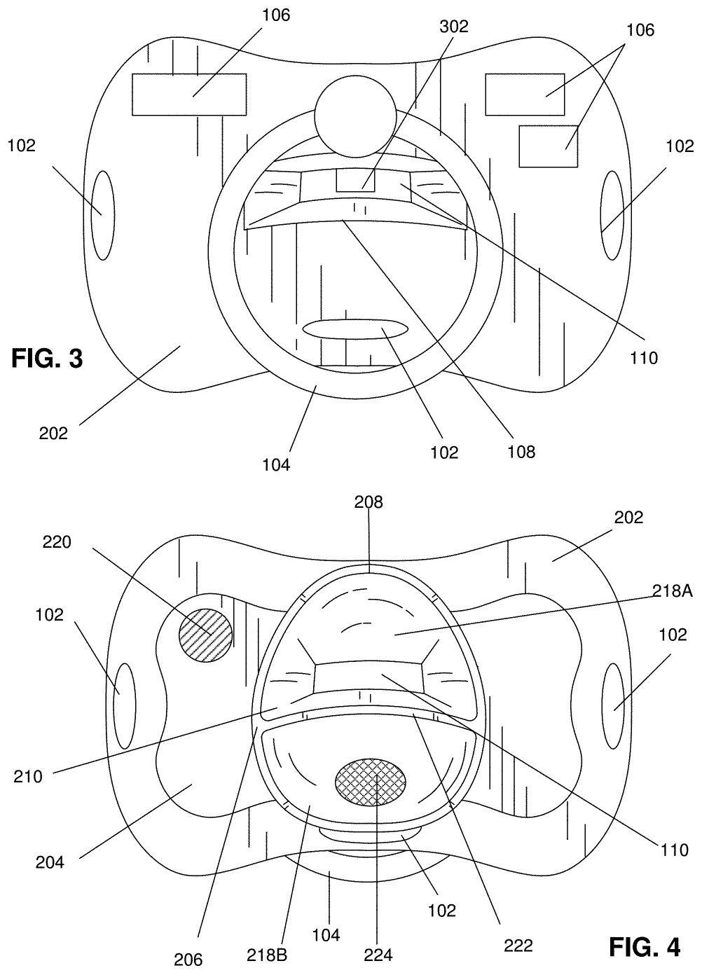

FIG. 3 and FIG. 4 illustrate the front view and back view, respectively, of respiratory airway assistive device 100, according to one embodiment of the present invention. As illustrated respiratory airway assistive device 100 can include airway maintenance body 206 that includes a dorsal (outer) surface 214 and an ventral (inner) surface 216, a first end 208, a first aperture 210 having a first opening positioned at or near the first end, a second end 301, a second aperture 108 positioned at or near the second end 301 and that is in fluidic communication with the first aperture 210 through fluidic communication channel 110. As illustrated, first end 208 is configured to be accommodated inside the mouth of user 101, and second end 301 is configured to be outside the oral cavity of user 101. The first end 208 includes a curvature on a ventral surface at distal ends of airway maintenance body 206, the curvature at the ventral surface configured to be included with an oral cavity of user 101. In one embodiment, the dorsal surface of respiratory airway assistive device 100 is configured to come in direct contact with the palate of the oral cavity of user 101 at a superior end and the floor of the oral cavity at an inferior end. In one embodiment, intraoral cavity 218A comes in contact with second aperture 210 to communicate with first aperture 108 (and the external environment) through fluidic communication channel 110.

In one embodiment, respiratory airway maintenance device 100 includes an intraoral cavity and is segmented by divider 222 into an upper intraoral space 218A and lower intraoral space 218B. Upper intraoral space 218A of respiratory airway maintenance device 100 can be configured to accommodate first aperture 210 and provides an open airway to assist in an obstruction free fluidic communication via fluidic communication channel 110 with second aperture 210. In one embodiment, lower intraoral space 218B can be configured as a tongue stabilizer to accommodate the tongue of user 101. In one or more implementations, respiratory airway maintenance device 100 can also include temperature sensor 224. Temperature sensor 224 can be a sublingual thermometer. respiratory airway assistive device 100 can also include vitals monitoring sensor 220 to measure the vitals of user 101 and can be configured to be located at a surface of the vestibular guard.

A dorsal surface of respiratory airway assistive device 100 can also include at least one of a vitals display, temperature display, and a dosage timer display. In one embodiment, the mentioned displays can be accommodated on a dorsal surface of lip guard 202 for easy visual reading and monitoring.

Sound magnifier 302 can be included within fluidic communication channel 110, as illustrated. In one embodiment, sound magnifier 302 can be positioned near or at second (outer) aperture 108 within fluidic communication channel 110. In yet another embodiment, respiratory airway assistive device 100 can also include sound magnifier 302 to vibrate as air current runs through fluidic communication channel 110. Sound magnifier 302 can be located in the path of aeration hole, within or near fluidic communication channel. Vibrations of sound magnifier 302 can generate an audible sound (e.g., whistling sound) and provide an indicator that gaseous exchange is occurring by inhale and exhale. This embodiment, with sound magnifier 302, is useful for toddlers and can provide parents a peace of mind that their toddler is able to breath without obstruction in fluidic communication channel 110.

In yet another embodiment, sound magnifier can have a vibration sensor that can detect if an obstruction is being caused in fluidic communication channel (to detect whether proper breathing is occurring). In this embodiment, vibrator sensor can then transmit a visual or audible signal in case any errors are detected. Thus, in this embodiment, visual display 106 can include the capacity to include auditory or visual indicators to provide the alarm of vibrator sensor.

As illustrated, respiratory airway assistive device 100 can also be configured to accommodate a retaining strap affixed to the lip guard via perforations 102. Respiratory airway assistive device 100 can further include a pulse oximeter. In one embodiment, a dorsal surface of the respiratory airway maintenance body 206 can be configured to not contact teeth or a gingiva of the oral cavity of user 101.

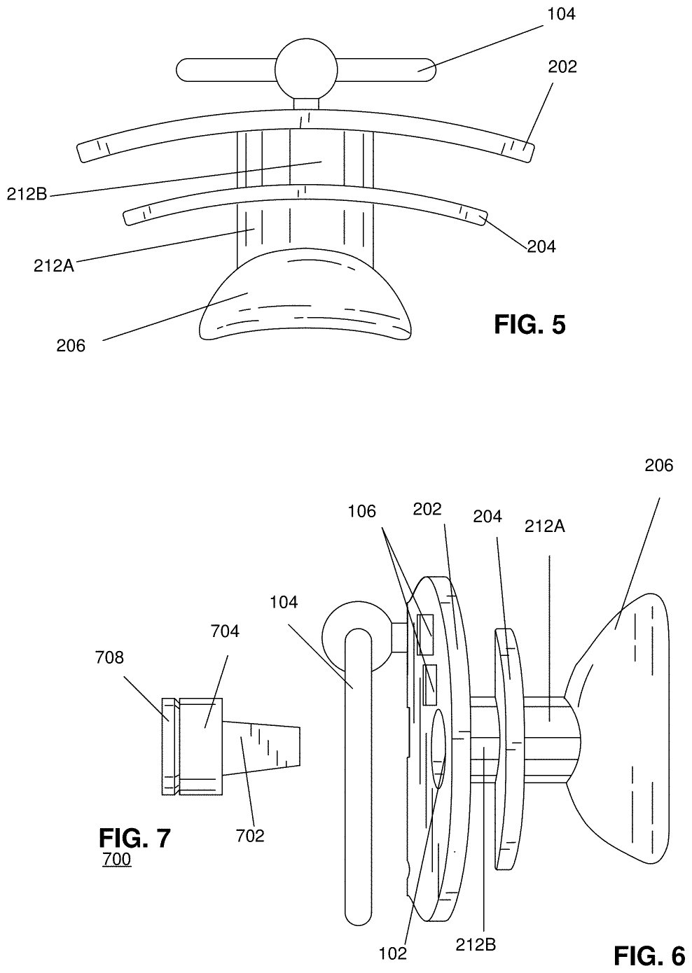

FIG. 5 and FIG. 6 illustrate the top view and a side view of respiratory airway assistive device 100, according to one embodiment of the present invention. As illustrated, respiratory airway assistive device 100 includes vestibular guard 204 coupled to airway maintenance body 206 forming an occlusal rest portion on a dorsal surface, at a distal end, of airway maintenance body 206. Respiratory airway assistive device 100 also includes lip guard 202 coupled to the airway maintenance body, via fluidic communication channel 110, forming an lip rest portion 212B on a dorsal surface of respiratory airway assistive device 100. Fluidic communication channel 110 of respiratory airway assistive device 100 can be configured to provide a clear airway from the second aperture 210 to the first aperture 108.

In yet another embodiment, airway maintenance body 206 comprises a flavorant. In yet another embodiment, the flavorant can be added in a packet or pouch (not shown) on airway maintenance body 206 that can be configured to be released in a timely manner. The flavorant can be medical grade and approved for oral consumption by a governmental regulatory agency (e.g., the Food and Drug Administration (FDA)).

In one embodiment, airway maintenance body 206 is dimensioned to fit within the oral cavity of a child of nursing age. respiratory airway assistive device 100 can be of pliant material and can be constructed from rubber, latex, polyurethane, vinyl, silicone rubber, nitrile, or a combination thereof. In one embodiment, respiratory airway assistive device 100 is construed using medical grade materials.

FIG. 7 illustrates a connector (or coupling) that can be configured to connect with respiratory airway assistive device 100, according to one embodiment of the present invention. As illustrated, in one embodiment, respiratory airway assistive device 100 comprises a coupling (connector) 700 that is in fluidic communication with first aperture 210 via fluidic communication channel 110. Coupling 700 can include connection portion 702 that can be configured to mate and couple with aperture 110. Coupler 700 can also include a coupling body (cavity) 704 that provides (and assists) fluidic communication from an external source (e.g., ventilator). In one embodiment, coupling 700 and can be configured to mate with a ventilator or any external device with connection portion 708 that can be configured according to the specifications required by the external source. Coupling 700 can thus assist user 101 with respiration by coupling respiratory airway assistive device 100 with the external source.

All the features or embodiment components disclosed in this specification, including any accompanying abstract and drawings, unless expressly stated otherwise, may be replaced by alternative features or components serving the same, equivalent or similar purpose as known by those skilled in the art to achieve the same, equivalent, suitable, or similar results by such alternative feature(s) or component(s) providing a similar function by virtue of their having known suitable properties for the intended purpose. Thus, unless expressly stated otherwise, each feature disclosed is one example only of a generic series of equivalent, or suitable, or similar features known or knowable to those skilled in the art without requiring undue experimentation.

Having fully described at least one embodiment of the present invention, other equivalent or alternative methods of implementing the invention described herein will be apparent to those skilled in the art. Various aspects of the invention have been described above by way of illustration, and the specific embodiments disclosed are not intended to limit the invention to the particular forms disclosed.

The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Although the present invention has been described with reference to specific exemplary embodiments, it will be evident that various modifications and changes may be made to these embodiments without departing from the broader spirit and scope of the invention as set forth in the claims. Accordingly, the specification and drawings are to be regarded in an illustrative rather than a restrictive sense.

1. A respiratory airway assistive device, comprising:
an airway maintenance body comprising an dorsal surface and an ventral surface, a first end, a first aperture having a first opening positioned at or near the first end, a second end, a second aperture positioned at or near the second end and that is in communication with the first aperture via a fluidic communication channel, wherein the first end is configured to be accommodated inside the oral cavity of a human subject, and wherein the second end is configured to be outside the oral cavity, and wherein the first end includes a curvature, the curvature at the ventral surface accommodating a cavity; and
a vestibular guard coupled to the airway maintenance body forming an occlusal rest portion on the dorsal surface or the airway maintenance body;
wherein the respiratory airway assistive device is constructing with a plaint material.

2. The respiratory airway maintenance device of claim 1, wherein the dorsal surface comes in direct contact with a palate of the oral cavity of the human subject at a superior end and a floor of the oral cavity at an inferior end.

3. The respiratory airway maintenance device of claim 1, wherein the cavity is segmented into an upper intraoral space and a lower intraoral space.

4. The respiratory airway maintenance device of claim 3, wherein the upper intraoral space accommodates the first aperture and provides an open airway to assist in an obstruction free fluidic communication with the second aperture, and the lower intraoral space is configured as a tongue stabilizer to accommodate the tongue of the human subject.

5. The respiratory airway maintenance device of claim 1, further including a temperature sensor.

6. The respiratory airway maintenance device of claim 5, wherein the temperature sensor is a sublingual thermometer.

7. The respiratory airway maintenance device of claim 1, further including a vitals monitoring sensor to monitor the vitals of the human subject.

8. The respiratory airway maintenance device of claim 7, wherein the vitals monitoring sensor is configured to be located on a surface of the vestibular guard.

9. The respiratory airway maintenance device of claim 1, further comprising a lip guard coupled to the airway maintenance body forming a lip rest portion on the dorsal surface of the airway maintenance body, and wherein a dorsal surface of the lip guard includes at least one of a vitals display, temperature display, and a timer.

10. The respiratory airway maintenance device of claim 1, wherein the fluidic communication channel includes a sound magnifier.

11. The respiratory airway maintenance device of claim 10, wherein the sound magnifier is configured to be positioned near or at the second aperture within the fluidic communication channel.

12. The respiratory airway assistive device of claim 9, configured to accommodate a retaining strap affixed to the lip guard.

13. The respiratory airway assistive device of claim 1, further comprising a pulse oximeter.

14. The respiratory airway assistive device of claim 1, further comprising a coupling that is in fluidic communication with the second aperture.

15. The respiratory airway assistive device of claim 14, wherein the coupling is configured to mate with a ventilator.

16. The respiratory airway assistive device of claim 1, wherein the airway maintenance body is dimensioned to fit within the oral cavity of a child of nursing age.

17. The respiratory airway assistive device of claim 1, wherein the pliant material is at least one of rubber, latex, polyurethane, vinyl, silicone rubber, or nitrile.

18. The respiratory airway maintenance device of claim 1, wherein the fluidic communication channel is configured to provide a clear airway from the second aperture to the first aperture.

19. The respiratory airway assistive device of claim 1, wherein the airway maintenance body comprises a flavorant.

20. The respiratory airway assistive device of claim 1, wherein the dorsal surface of the respiratory airway maintenance body is configured so as to not contact teeth or a gingiva of the oral cavity when in use.
